(19) United States
(12) Patent Application Publication
(10) Pub. No.: US 2019/0125302 A1
(43) Pub. Date: May 2, 2019

(54) ACCELEROMETER IN HANDLE FOR ULTRASOUND MEDICAL IMAGING DEVICE

(71) Applicant: KONINKLIJKE PHILIPS N.V., Einhoven (NL)

(72) Inventor: David Wesley Clark, Derry, NH (US)

(21) Appl. No.: 16/165,604

(22) Filed: Oct. 19, 2018

Related U.S. Application Data
(60) Provisional application No. 62/579,336, filed on Oct. 31, 2017.

(51) Int. Cl. A61B 8/00; A61B 8/12; A61B 8/08

(52) U.S. Cl. CPC A61B 8/4254; A61B 8/12; A61B 8/463; A61B 8/0883; A61B 8/445; A61B 8/4466; A61B 8/5238; A61B 8/4488

(57) ABSTRACT

Ultrasound imaging devices, systems and methods are provided. In some embodiments, the ultrasound imaging devices include a flexible elongate member including a proximal portion and a distal portion, the distal portion configured to be positioned within a body of a patient, an ultrasound imaging element disposed at the distal portion of the flexible elongate member and configured to obtain imaging data from within the body of the patient; and a handle coupled to the proximal portion of the flexible elongate member. The handle includes an accelerometer configured to determine an orientation of the ultrasound imaging element.

### RELATED APPLICATION

This application claims the benefit of and priority to U.S. Provisional Application No. 62/579,336, filed Oct. 31, 2017, which is incorporated by reference in its entirety.

### TECHNICAL FIELD

The present disclosure relates generally to ultrasound imaging devices and systems. In particular, the present disclosure provides an ultrasound imaging device with an accelerometer-embedded handle that determines orientation of the ultrasound transducer.

### BACKGROUND

Diagnostic and therapeutic ultrasound catheters have been designed for use inside many areas of the human body. For example, a trans-esophageal echocardiography (TEE) probe can be inserted into an esophagus of a patient and image the patient's heart with high frequency ultrasound waves. Another common diagnostic ultrasound method is intra-cardiac echocardiography (ICE), where a single rotating transducer or an array of transducer elements (sometimes referred to as a transducer array) is threaded through a patient's femoral vein or jugular vein and enters his or her heart to take images. Transducers on TEE probes and ICE catheters transmit ultrasound waves and receive echoes from the tissue. A signal generated from the echoes is transferred to a console which allows for the processing, storing, display, or manipulation of the ultrasound-related data.

TEE probes and ICE catheters are usually used along with a radiographic imaging device, such as a fluoroscopy system, to guide and facilitate medical procedures, such as transseptal lumen punctures, left atrial appendage closures, atrial fibrillation ablation, and valve repairs. In order to co-register the ultrasound images from a TEE probe or an ICE catheter and radiographic images, a computer system has to identify the position and orientation of the ultrasound transducer. In case of TEE, because the diameter of a TEE probe can be as large as 13 mm, the TEE probe and its internal structure can be discerned by the computer system in the radiographic images. However, in case of a mini TEE probe or an ICE catheter that can have a diameter of about 3 mm, while the computer system can determine its position, its rotational orientation may not be detectable with adequate precision in the radiographic images. Without reliable information of the orientation, co-registration may fail or lose fidelity.

### SUMMARY

Embodiments of the present disclosure provide an ultrasound imaging system having a handle containing an accelerometer. For example, the ultrasound imaging system can be an intracardiac echocardiography (ICE) catheter that obtains ultrasound images from within the chambers of the heart or any other suitable ultrasound device. An ultrasound imaging element is positioned at the distal portion of the imaging device, which is disposed within the body of the patient to obtain ultrasound images. The handle is positioned at the proximal portion of the imaging device. A doctor controls where the ultrasound images are obtained using the handle. For example, the doctor can rotate the handle to correspondingly rotate the imaging element. The accelerometer monitors the orientation of the handle, and thus the corresponding orientation of the imaging element, and where in the patient's anatomy the ultrasound images are being obtained. The orientation data from the accelerometer can be used, e.g., together with an X-ray image of the patient's anatomy, to identify which part of the anatomy is shown in the ultrasound images. Advantageously, the accelerometer can provide orientation data when the ultrasound imaging element is small such that its orientation cannot be determined precisely enough by looking at the X-ray image itself. The medical imaging systems described herein reliably determine the orientation of the imaging element of the ultrasound imaging device. The orientation data is particularly advantageous when registering internal imaging data with external imaging data, such as angiograms or x-ray imaging data.

In one embodiment, an ultrasound imaging system is provided. The ultrasound imaging system includes a flexible elongate member having a proximal portion and a distal portion, the distal portion configured to be positioned within a body of a patient, an ultrasound imaging element disposed at the distal portion of the flexible elongate member and configured to obtain imaging data from within the body of the patient; and a handle coupled to the proximal portion of the flexible elongate member. The handle includes an accelerometer configured to determine an orientation of the ultrasound imaging element. The system may further include a computing device in communication with the handle and the ultrasound imaging element, wherein the computing device is configured to receive the ultrasound and orientation data and co-register the received data to radiographic imaging data.

In some implementations, the accelerometer is a dual-axis accelerometer. In some instances, the ultrasound imaging element includes a transducer array. In some embodiments, the handle of the ultrasound imaging device includes a first actuator, which, upon activation, is configured to deflect the distal portion of the flexible elongate member along a first plane. In still some embodiments, the handle of the ultrasound imaging device includes a second actuator, which, upon activation, is configured to deflect the distal portion of the flexible elongate member along a second plane not parallel to the first plane

In another embodiment, a medical imaging system is provided. The method includes an ultrasound imaging device configured to obtain ultrasound imaging data of an area of interest within a body of a patient and a computing device in communication with the ultrasound imaging device and a radiographic imaging device. The ultrasound imaging device includes a flexible elongate member and a handle. A distal portion of the flexible elongate member is sized and shaped to be inserted into the body and placed in the area of interest. The handle includes an accelerometer configured to determine an orientation of the flexible elongate member. The computing device is configured to receive radiographic imaging data of the area of interest from the radiographic imaging device; determine a position of the ultrasound imaging device using the radiographic imaging data; receive the orientation of the flexible elongate member from the accelerometer; receive ultrasound imaging data from the ultrasound imaging device; co-register the ultrasound imaging data and the radiographic imaging data; and output the co-registered ultrasound imaging data and radiographic imaging data to a display. In some embodiments, the computing device is further configured to control the ultrasound imaging device and the radiographic imaging device. In some implementations, the computing device is further configured to overlay the ultrasound imaging data over the radiographic imaging data co-registered with the ultrasound imaging data and output to the display the ultrasound imaging data overlaid over the radiographic imaging data co-registered with the ultrasound imaging device. In some instances, the medical imaging system further includes the radiographic imaging device. In some implementations, the medical imaging system further includes the display. In some embodiments, the ultrasound imaging device is intra-cardiac echocardiography (ICE) device. In some other embodiments, the ultrasound imaging device is a trans-esophageal echocardiography (TEE) device. In still other embodiments, the ultrasound imaging device is an intravascular ultrasound (IVUS) device. In some instances, the accelerometer is a dual-axis accelerometer. In some other instances, the accelerometer is a triple-axis accelerometer.

In another embodiment, a method for displaying ultrasound imaging data and radiographic imaging data with respect to an area of interest within a body of a patient is provided. The method includes obtaining the radiographic imaging data of the area of interest from outside of the body by a radiographic imaging device while an ultrasound imaging device is placed within the area of interest; determining a position of the catheter based on the radiographic imaging data; receiving the orientation of the ultrasound imaging device from the accelerometer; obtaining, by the ultrasound imaging device, the ultrasound imaging data of the area of interest from within the body; co-registering the ultrasound imaging data and the radiographic imaging data based on the position and rotational orientation of the ultrasound imaging device; and displaying the co-registered ultrasound imaging data and radiographic imaging data on a display. The ultrasound imaging device includes a handle that includes an accelerometer configured to determine an orientation of the ultrasound imaging device. In some embodiments, the method further includes overlaying ultrasound imaging data over the radiographic imaging data co-registered with the ultrasound imaging data. In some implementations, the method further includes displaying the ultrasound imaging data overlaid over the radiographic imaging data co-registered with the ultrasound imaging data.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a picture of a radiographic image showing a TEE probe and a catheter within a patient's body.

FIG. 2 is a schematic diagram of a medical imaging system according to embodiments of the present disclosure.

FIG. 3 is a perspective view of an imaging assembly of an ultrasound imaging device in two substantially opposing orientations.

FIG. 4 is a schematic diagram illustrating a portion of an ultrasound imaging device under deflection according to embodiments of the present disclosure.

FIG. 5 is a schematic diagram illustrating an ultrasound imaging device with an imaging assembly positioned on a distal portion thereof inserted into a heart in a first orientation and a proximal portion thereof coupled to a handle, according to embodiments of the present disclosure.

FIG. 6 is a schematic diagram illustrating the ultrasound imaging device with the imaging assembly inserted into a heart in a second orientation different from the first direction and the proximal portion coupled to the handle, according to embodiments of the present disclosure.

FIG. 7 is a flow diagram of a method for displaying ultrasound imaging data and radiographic imaging data with respect to an area of interest within a body of a patient, according to aspects of the disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, while the ICE system is described in terms of intraluminal imaging, it is understood that it is not intended to be limited to this application. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

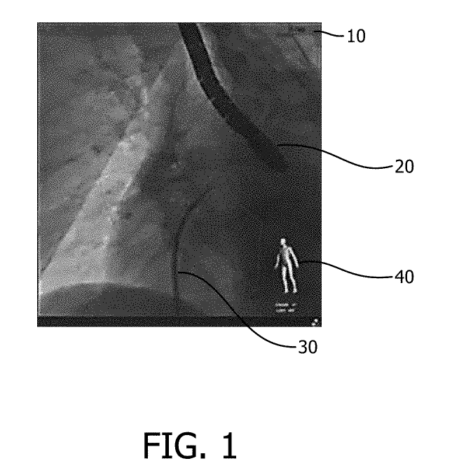

FIG. 1 is a picture of a radiographic image 10 showing a TEE probe 20 and a catheter 30 within a patient's body to image an area of interest. In this case, the area of interest is within the patient's heart. In some instances, the radiographic image 10 is captured by a radiography imaging device, such as a fluoroscopy system and is displayed on a display device. The TEE probe 20 is inserted into the patient's esophagus through the patient's mouth. Sometimes, if the circumstances require, the TEE probe 20 can be inserted all the way into the patient's stomach to image the patient's heart at a preferable angle. A catheter 30 enters the patient's body through his or her femoral or jugular vein and is threaded to the patient's heart. When a catheter such as catheter 30 enters the patient's body through his/her jugular vein, the catheter 30 is advanced downward into the patient's heart through the superior vena cava. However, when catheter 30 enters the patient's body through his/her femoral vein, the catheter would be advanced upward into the patient's heart through the inferior vena cava.

As illustrated in FIG. 1, the radiographic imaging device that captures the radiographic image 10 is directed at the area of interest from outside of the patient's body while the TEE probe 20 and the catheter 30 is positioned adjacent to or within the patient's heart. Due to the difference in dimensions of the esophagus and vein that allow passage of a regular TEE probe, such as the TEE probe 20, and catheter 30, the TEE probe 20 has a diameter substantially larger than that of the catheter 30. External characteristics of the TEE probe 20 can be readily identified by a computer system that processes radiographic images from the radiographic imaging device. In some implementations, X-ray from the radiographic imaging device penetrates certain outer radiolucent features of the TEE probe 20 and allows the computer system to discern inner radiopaque structures of the TEE probe 20. Because the computer system can identify features and characteristics of the TEE probe 20, the computer system can determine a position and an orientation of the TEE probe 20. The same cannot be said for the catheter 30. Given the resolution of the radiographic imaging data, while the position of catheter 30 is readily identifiable, the smaller dimensions of catheter 30 may not provide enough discernible features and characteristics for the computer system to determine the orientation of catheter 30. In some instances, the orientation refers to the rotational orientation around a longitudinal direction along the length of catheter 30. For example, the silhouette of the catheter 30 in the radiographic image 10 provides little details. In contrast, the silhouette of the TEE probe 20 reveals much more details, including the gaps between vertebrae embedded within a polymer jacket. In some examples, a figurine icon 40 is also displayed to indicate the imaging plane of the displayed radiographic image relative to the patient's body.

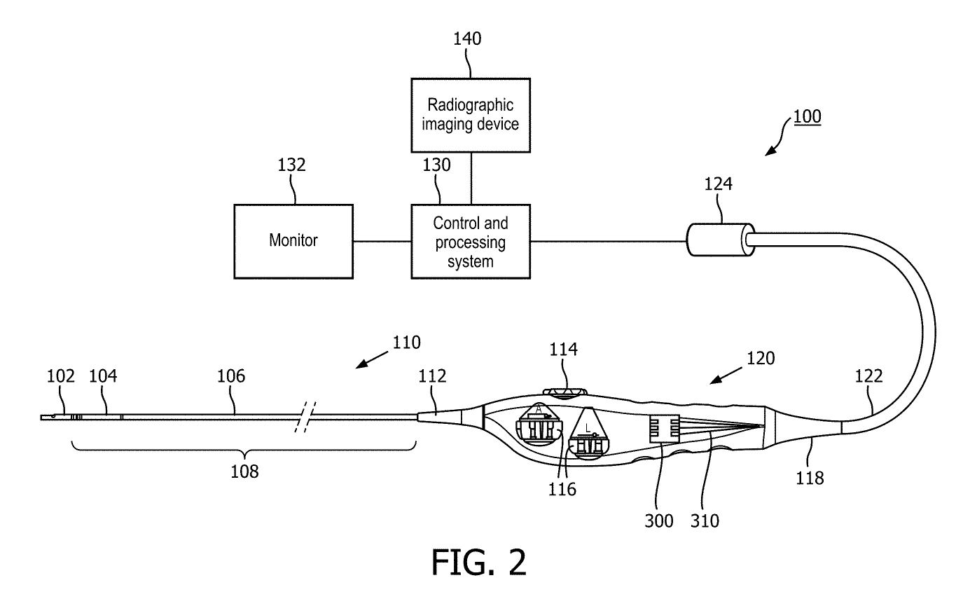

FIG. 2 shows a schematic diagram of a medical imaging system 100 according to embodiments of the present disclosure. The system 100 can include an ultrasound imaging device 110, a connector 124, a control and processing system 130 (for example, a console and a computer), and a monitor 132. In some embodiments, the ultrasound imaging device 110 is an ICE catheter. The ultrasound imaging device 110 includes an imaging assembly 102 at the tip of a flexible elongate member 108, and a handle 120. The flexible elongate member 108 includes a distal portion 104 and a proximal portion 106. The distal end of the distal portion 104 is attached to the imaging assembly 102. The proximal end of the proximal portion 106 is attached to the handle 120, for example, by a resilient strain reliever 112. The handle 120 may be used for manipulation of the ultrasound imaging device 110 and manual control of the ultrasound imaging device 110. The imaging assembly 102 can include an imaging core with ultrasound transducer elements and associated circuitry. The handle 120 can include actuators 116, a clutch 114, and other steering control components for steering the ultrasound imaging device 110. The steering may include deflecting the imaging assembly 102 and the distal portion 104 along different planes, as described in greater details herein.

The handle 120 is connected to the connector 124 via another strain reliever 118 and a connection cable 122. The connector 124 may be configured to provide suitable configurations for interconnecting the control and processing system 130 and the monitor 132 to the imaging assembly 102. The control and processing system 130 may be used for processing, storing, analyzing, and manipulating data, and the monitor 132 may be used for displaying obtained signals generated by the imaging assembly 102. The control and processing system 130 can include one or more processors, memory, one or more input devices, such as keyboards and any suitable command control interface device. The control and processing system 130 can be operable to facilitate the features of the medical imaging system 100 described herein. For example, a processor can execute computer readable instructions stored on the non-transitory tangible computer readable medium. The monitor 132 can be any suitable display device, such as liquid-crystal display (LCD) panel or the like.

In some embodiments, the handle 120 includes an accelerometer 300 coupled to a plurality of electrical wires 310. In some instances, the accelerometer 300 is a dual-axis accelerometer. In some other examples, the accelerometer 300 is a triple-axis accelerometer. As compared to a dual-axis accelerometer that measures acceleration along an X and a Y axes, a three-axis or triple-axis accelerometer measures acceleration along an additional Z axis. In embodiments where a triple-axis accelerometer is used and its Z-axis is aligned with the longitudinal direction of the handle 120, the tilting movement of the handle 120 can be measured. In some embodiments, the accelerometer 300 is a micro-machined accelerometer that is integrated with a signal conditioning circuit. The accelerometer 300 is positioned inside of handle 120 to measure accelerations along at least two axes of the handle 120 relative to the vertical force of earth's gravity. This way, the accelerometer 300 is able to detect and measure rotational orientation of the handle 120 for a dual-axis accelerometer, and also tilting for a triple-axis accelerometer. In some instances, as the flexible elongate member 108 is torsionally stiff and attached to the handle 120, the rotational orientation of the handle 120 assumes a one-to-one correlation with the rotational orientation of the flexible elongate member 108. Because the imaging assembly 102 is attached to the distal portion 104 of the flexible elongate member 108, the rotational orientation of the imaging assembly 102 also assumes a one-to-one correlation with the rotation orientation of the handle 120. As a result, in some embodiments, the accelerometer 300 measures and determines the rotational orientation of the imaging assembly 102 by measuring and determining the rotational orientation of the handle 120. The accelerometer 300 transmits orientation data, including the rotational orientation of the handle 120 and the imaging assembly 102, to the control and processing system 130 by way of electrical wires 310.

In some embodiments, a dual-axis accelerometer 300 can be replaced by two single-axis accelerometers. Similarly, a triple-axis accelerometer 300 can be replaced by two dual-axis accelerometers or three single-axis accelerometers. Further, in some other embodiments, the accelerometer 300 can be replaced by a gyroscope. The gyroscope can be a single-axis, a dual-axis or a triple-axis gyroscope. In some embodiments, one or both of an accelerometer or a gyroscope can be implemented in the handle 120. A gyroscope positioned inside the handle 120 can measure the rotational orientation of the handle 120 and the rotational orientation of the imaging assembly 102, provided that the flexible elongate member 108 connecting the handle 120 and the imaging assembly 102 is torsionally stiff

The electrical wires 310 have distal ends and proximal ends. In some embodiments, the distal ends of the electrical wires 310 are bonded to bond pads on the accelerometer 300 or bond pads on a substrate on which the accelerometer 300 is mounted; and the proximal ends of the electrical wires 310 are connected to the control and processing system 130 by way of the connection cable 122 and the connector 124. In some instances, the accelerometer 300 or the substrate, on which the accelerometer 300 is mounted, is mounted or coupled to the interior of the handle 120. Arranged in that fashion, each of the actuators 116 is attached to one or more distally-extending pull wires that are threaded through the flexible elongate member 108 and anchored to the distal portion 104; and the accelerometer 300 is bonded to the plurality of electrical wires 310 that extend proximally and enter the connection cable 122.

In operation, a physician or a clinician may advance the flexible elongate member 108 into a vessel within a heart anatomy. By controlling the actuators 116 and the clutch 114 on the handle 120, the physician or clinician can steer the flexible elongate member 108 to a position near the area of interest to be imaged. For example, one actuator 116 may deflect the imaging assembly 102 and the distal portion 104 in along a first plane and the other actuator 116 may deflect the imaging assembly 102 and the distal portion 104 along a second plane not parallel to the first plane. In some embodiments, the first plane has a normal direction perpendicular to the normal direction of the second plane. The clutch 114 provides a locking mechanism to lock the positions of the actuators 116 and in effect lock the deflection of the flexible elongate member while imaging the area of interest. Embodiments of the present disclosure can include steering mechanism features similar to those described in U.S. Provisional App. No. 62/402,483, filed Sep. 30, 2016, the entirety of which is hereby incorporated by reference herein. Additionally, embodiments of the present disclosure can include features similar to those described in U.S. Provisional App. No. 62/403,479, filed Oct. 3, 2016, U.S. Provisional App. No. 62/434,517, filed Dec. 15, 2016, U.S. Provisional App. No. 62/403,311, filed Oct. 3, 2016, U.S. Provisional App. No. 62/437,778, filed Dec. 22, 2016, U.S. Provisional App. No. 62/401,464, filed Oct. 29, 2016, U.S. Provisional App. No. 62/401,686, filed Oct. 29, 2016, and/or U.S. Provisional App. No. 62/401,525, filed Oct. 29, 2017, the entireties of which are hereby incorporated by reference herein.

The imaging process may include activating the ultrasound transducer elements on the imaging assembly 102 to produce ultrasonic energy. A portion of the ultrasonic energy is reflected or scattered by the area of interest and the surrounding anatomy, and the ultrasound echo signals are received by the ultrasound transducer elements. The connector 124 transfers the received echo signals in the form of ultrasound imaging data to the control and processing system 130 where the ultrasound image is reconstructed and displayed on the monitor 132. In some embodiments, the control and processing system 130 can control the activation of the ultrasound transducer elements and the reception of the echo signals. In some embodiments, the control and processing system 130 and the monitor 132 may be part of a same system. In some instances, the ultrasound image can be further displayed in a different monitor (not shown in FIG. 2) to be viewed by a physician or clinician.

The system 100 may be utilized in a variety of applications such as trans-septal punctures, left atrial appendage closures, atrial fibrillation ablation, and valve repairs and can be used to image vessels and structures within a living body. In some examples, the device 110 can be sized and shaped, structurally arranged, and/or otherwise configured to be positioned within any suitable anatomy and/or body lumen of a patient. For example, the device 110 can be an intraluminal device. Although the system 100 is described in the context of intraluminal imaging procedures, the system 100 is suitable for use with any catheterization procedure, e.g., ICE, mini TEE, or intravascular ultrasound (IVUS). More generally speaking, the system is suitable for use with any medical imaging procedures where the angular and/or rotational orientation of an imaging element is of interest and bears a pre-determined relationship with the angular and/or rotational orientation of a handle connected directly or indirectly to the imaging element. An example of such a medical imaging procedure includes external ultrasound examination where an external ultrasound imaging device is used. For example, disclosure described herein can be implemented for any ultrasound transducer with an accelerometer in the handle to determine orientation. In addition, the imaging assembly 102 may include any suitable physiological sensor or component for diagnostic, treatment, and/or therapy. For example, the imaging assembly can include an imaging component, an ablation component, a cutting component, a morcellation component, a pressure-sensing component, a flow-sensing component, a temperature-sensing component, and/or combinations thereof.

In some embodiment, the ultrasound imaging device 110 includes a flexible elongate member 108 that can be positioned within a vessel, such as a femoral vein or a jugular vein. The flexible elongate member 108 may have a distal portion 104 and a proximal portion 106. The ultrasound imaging device 110 includes an imaging assembly 102 that is mounted within the distal portion 104 of the flexible elongate member 108.

In some embodiments, the medical imaging system 100 is used for generating 2D and 3D images. In some examples, the medical imaging system 100 is used for generating multi-plane images in two or more different planes at some non-zero angle or distance to each other.

In some embodiments, the control and processing system 130 is in communication with a radiographic imaging device 140. In some embodiments, the radiographic imaging device 140 is a fluoroscopy system. In some other embodiments, the radiographic imaging device 140 is an X-ray imaging device other than a fluoroscopy system. The control and processing system 130 can be used to activate, operate, and control the radiographic imaging device 140. In some embodiments, the control and processing system 130 may be used for processing, storing, analyzing, and manipulating radiographic imaging data received from the radiographic imaging device 140, and the monitor 132 may be used for displaying obtained radiographic imaging data generated by the radiographic imaging device 140. The control and processing system 130 can include one or more processors, memory, one or more input devices, such as keyboards and any suitable command control interface device. The control and processing system 130 can be operable to facilitate the features of the medical imaging system 100 described herein. For example, a processor can execute computer readable instructions stored on the non-transitory tangible computer readable medium.

In some embodiments, the control and processing system 130 outputs both ultrasound imaging data received from the ultrasound imaging device 110 and the radiographic imaging data received from the radiographic imaging device 140, to the monitor 132 for simultaneous display of the ultrasound imaging data and the radiographic imaging data. In some instances, the ultrasound imaging data and the radiographic imaging data are displayed on the monitor 132 in real time while the ultrasound imaging device 110 and the radiographic imaging device 140 are capturing them within the patient's body.

In some embodiments, the ultrasound imaging data and the radiographic imaging data are co-registered before they are displayed on the monitor. To co-register the ultrasound imaging data and the radiographic imaging data, the control and processing system 130 has to identify the position and orientation of the ultrasound imaging device 110 and the radiographic imaging device 140. The orientation, field of view, and position of the radiographic imaging device 140 is readily available to the control and processing system 130 as the movement of the radiographic imaging device 140 is controlled through the control and processing system 130. In some embodiments where fluoroscopy system is used, the radiographic imaging element orbits around the patient's body along a C-shaped arm (C-arm) and the orbiting is controlled through the control and processing system 130.

With respect to the position of the ultrasound imaging device 110, the control and processing system 130 can determine the position of the imaging assembly 102 based on radiographic imaging data obtained by the radiographic imaging device 140. To achieve that, the radiographic imaging device 140 is directed at the area of interest from outside of the patient's body while the imaging assembly 102 of the ultrasound imaging device 110 is advanced into and positioned within the area of interest. In some examples, the area of interest is the patient's heart. The foregoing arrangement allows the radiographic imaging device 140 to obtain radiographic imaging data that contain information of the position of the imaging assembly 102. For example, such information includes distances between the imaging assembly 102 and two reference points adjacent to the area of interest within the patient's body. Oftentimes the imaging assembly 102 is too small for the control and processing system 130 to reliably determine the orientation of the imaging assembly 102. In some instances, the control and processing system 130 determines the orientation (for example, the rotational orientation) of the imaging assembly 102 based on orientation data from the accelerometer 300. Having obtained or received positions and orientations of the ultrasound imaging device 110 and the radiographic imaging device 140, the control and processing system 130 can co-register the ultrasound imaging data and the radiographic imaging data and output the co-registered imaging data to the monitor 132 for display. When displayed on the monitored 132, the co-registered imaging data are displayed such that the ultrasound image and the radiographic image share substantially the same field of view. In some embodiments, the control and processing system 130 can overlay the ultrasound image on top of the radiographic image to provide a more intuitive view for a physician or clinician performing the catheterization.

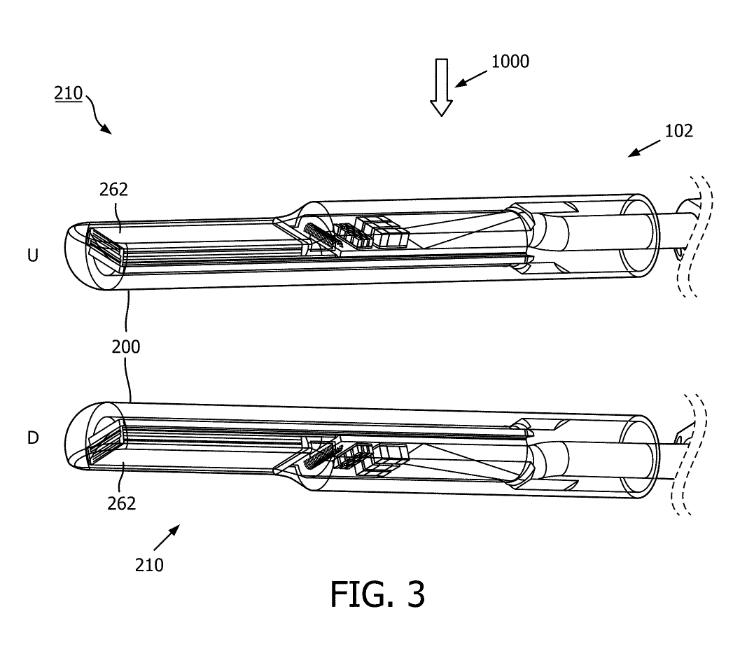

FIG. 3 is a perspective view of the imaging assembly 102 of the ultrasound imaging device 110 in two substantially opposing orientations. The imaging assembly 102 may include an imaging core 262 that is positioned within a tip member 200. In some embodiments, the tip member 200 is substantially cylindrical in shape except for the portion that houses the imaging core 262, which may be flat. In some embodiments, the imaging core 262 has a directional field of view 210. FIG. 3 shows the imaging assembly 102 in a “U” orientation where the imaging core 262 faces upward with an upward field of view 210 and another imaging assembly 102 in a “D” orientation where the imaging core 262 faces downward with a downward field of view 210. When being imaged along a direction 1000 by a radiographic imaging device such as the radiographic imaging device 140, because the tip member is cylindrical in shape and of a small diameter, the radiographic imaging data do not contain enough information for the control and processing system 130 to determine the rotational orientation of the imaging assembly 102 precisely enough to adequate co-register the images. Sometimes the small dimensions of the imaging assembly 102 can make it more difficult for the control and processing system 130 to distinguish the “U” and “D” orientations or any intermediate orientations. This makes it unreliable for the control and processing system 130 to determine the orientation of the imaging assembly 102 by the radiographic imaging data alone. Advantageously, the accelerometer 300 described in the present disclosure eliminates the unreliable orientation determination by radiographic imaging data and introduces fidelity in the orientation determination, thereby facilitating accurate and fast co-registration of the ultrasound imaging data and the radiographic imaging data.

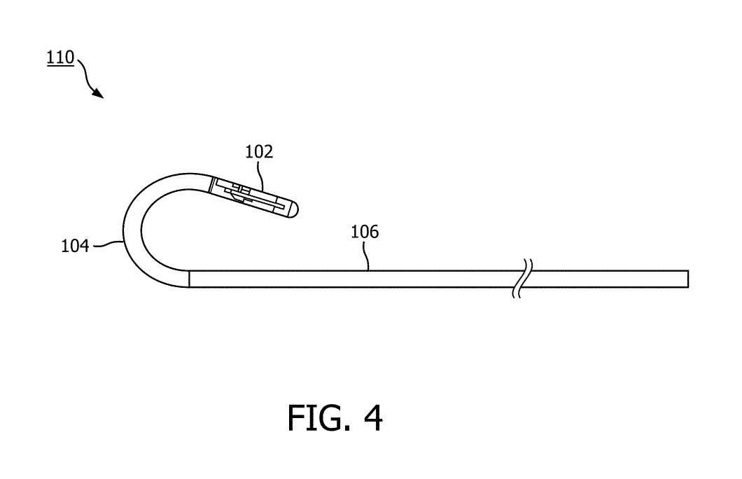

FIG. 4 is a schematic diagram illustrating a portion of the ultrasound imaging device 110 under deflection according to embodiments of the present disclosure. For example, the flexible elongate member 108 shown in FIG. 2 is referred to as a neutral position. In FIG. 4, the imaging assembly 102 and the distal portion 104 of the flexible elongate member 108 are deflected from the neutral position. As described above, the deflection is controlled by the actuators 116. The capability of the imaging assembly 102 to deflect allows the ultrasound imaging device 110 to obtain ultrasound imaging data of the area of interest from more directions. The deflection of the imaging assembly 102 is readily discernible by the control and processing system 130 from the radiographic imaging data obtained by the radiographic imaging device 140.

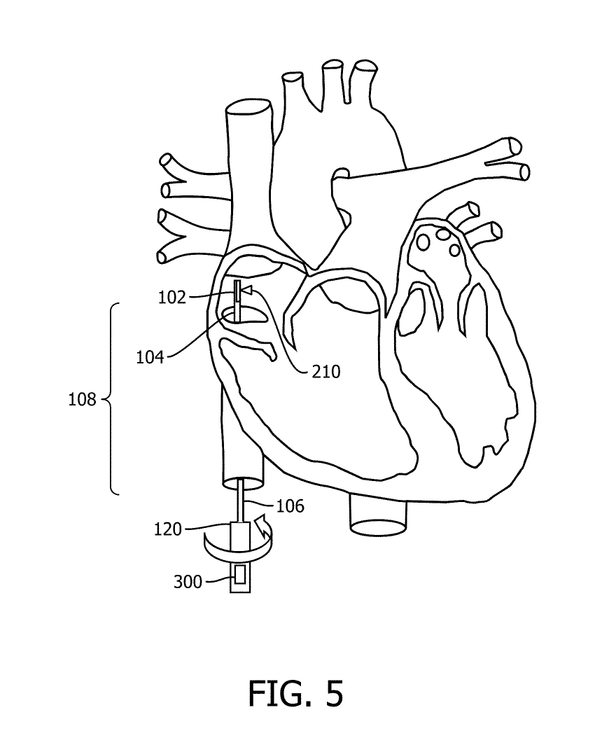

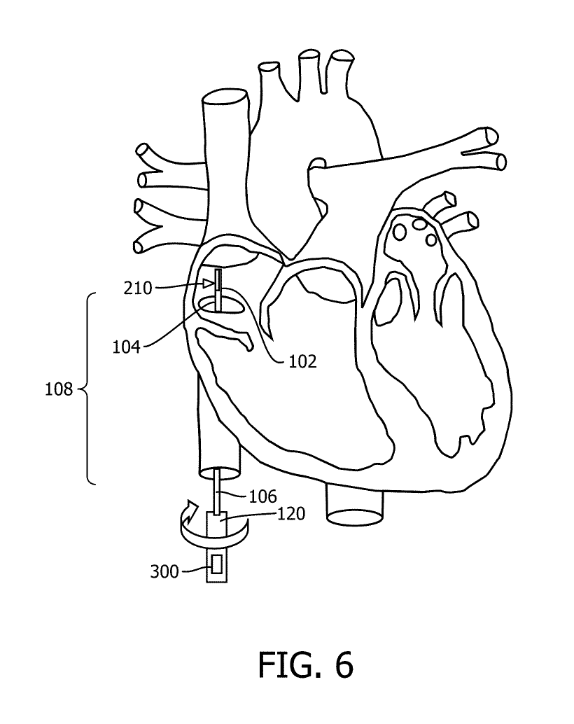

Reference is now made to FIGS. 5 and 6. FIG. 5 is a schematic diagram illustrating the ultrasound imaging device 110 with imaging assembly 102 positioned on distal portion 104 inserted into a heart of a patient in a first orientation and proximal portion 106 coupled to handle 120, according to embodiments of the present disclosure. When the imaging assembly 102 is in the first orientation, the field of view 210 of the imaging assembly 102 is pointed to the right of FIG. 5. FIG. 6 is a schematic diagram illustrating imaging assembly 102 being inserted into the heart in a second orientation different from the first orientation. When the imaging assembly 102 is in the second orientation, the field of view 210 of the imaging assembly 102 is pointed to the left of FIG. 6. As described above, because the flexible elongate member 108 is torsionally stiff, the flexible elongate member 108 and the imaging assembly 102 rotate with the handle 120. That is, by rotating the handle 120, the imaging assembly 102 can be rotated between the first orientation shown in FIG. 5 and the second orientation shown in FIG. 6. In some embodiments, the accelerometer 300 is mounted inside of the handle 120 and the accelerometer 300 determines the orientation of the handle 120, thereby determining the orientation of the flexible elongate member 108 and the imaging assembly 102. FIGS. 5 and 6 show that the imaging assembly 102 enters into the heart via the inferior vena cava, suggesting that the imaging assembly 102 is inserted into the patient's body through the femoral vein. In some other examples, the imaging assembly 102 can enter into the heart via the superior vena cava and the imaging assembly 102 is inserted into the patient's body through the jugular vein.

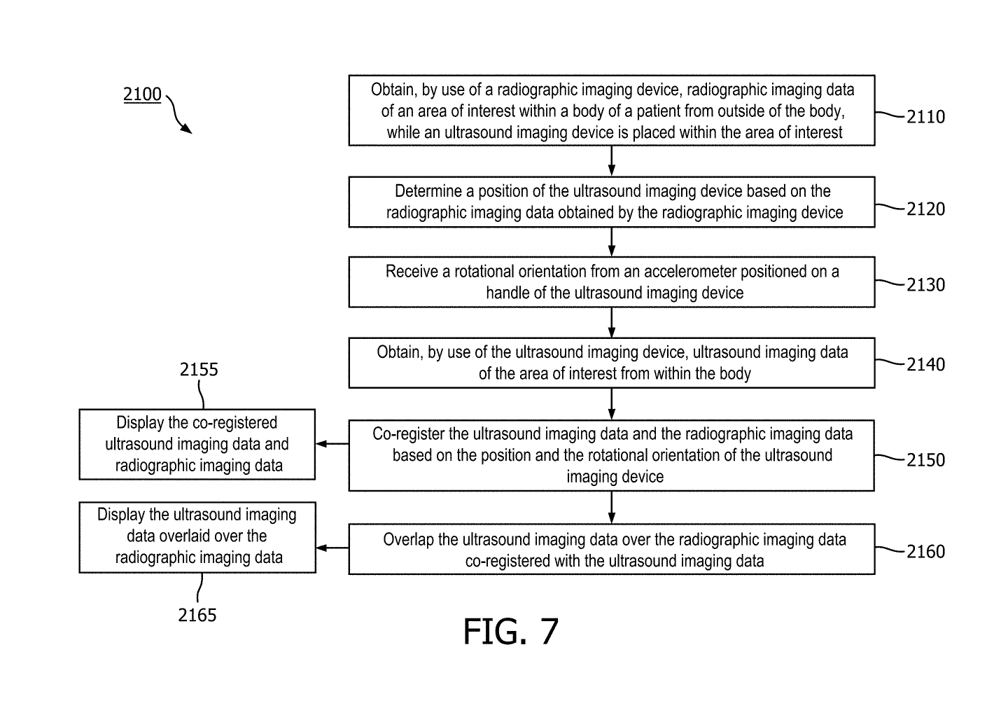

FIG. 7 is a flow diagram of a method 2100 for displaying ultrasound imaging data and radiographic imaging data with respect to the area of interest within a body of a patient, according to aspects of the disclosure. As illustrated, the method 2100 includes a number of enumerated steps, but embodiments of the method 2100 may include additional steps before, after, and in between the enumerated steps. In some embodiments, one or more of the enumerated steps may be omitted, performed in a different order, or performed concurrently. The method 2100 can be performed with reference to FIGS. 2, 5 and 6. At step 2110, radiographic imaging data of an area of interest within a body of a patient from outside of the body is obtained by the radiographic imaging device 140, while the imaging assembly 102 of the ultrasound imaging device 110 is placed within the area of interest. At this step, the radiographic imaging device 140 is directed at the area of interest from outside of the patient's body. Consequently, the radiographic imaging device 140 not only obtains radiographic imaging data of the area of interest, but also of the imaging assembly 102 within the area of interest. Therefore, the radiographic imaging data captured by the radiographic imaging device 140 contains imaging data about the position of the imaging assembly 102. In some embodiments, the flexible elongate member 108 is inserted into the patient's body and advanced into the patient's heart with assistance of another monitor separate from the monitor 132. In those embodiments, before the imaging assembly 102 is in position within the area of interest, the monitor 132 displays the radiographic imaging data obtained by the radiographic imaging device 140 and the other monitor displays the ultrasound imaging data obtained by the ultrasound imaging device 110.

At step 2120, the position of the imaging assembly 102 is determined by the control and processing system 130 based on the radiographic imaging data obtained by the radiographic imaging device 140. For example, the control and processing system 130 can determine the position of the imaging assembly 102 by determining the distances between the imaging assembly 102 and at least two reference points adjacent to the area of interest.

At step 2130, a rotational orientation is received from the accelerometer 300. Because the accelerometer 300 is positioned on the handle 120 of the ultrasound imaging device 110 and the flexible elongate member 108 is torsionally stiff, the rotational orientation from the accelerometer 300 represent not only the orientation of the handle 120, but also the orientation of the flexible elongate member 108 and the imaging assembly 102.

At step 2140, ultrasound imaging data of the area of interest are obtained by the ultrasound imaging device 110. At step 2150, the ultrasound imaging data obtained by the ultrasound imaging device 110 and the radiographic imaging data obtained by the radiographic imaging device 140 are co-registered based on the position and the rotational orientation of the ultrasound imaging device 110. At step 2155, the co-registered ultrasound imaging data and the radiographic imaging data are displayed on the monitor 132.

At step 2150, the ultrasound imaging data is overlaid over the radiographic imaging data that are co-registered with the ultrasound imaging data. At step 2165, the ultrasound imaging data overlaid over the radiographic imaging data are displayed on the monitor 132. In some embodiments, the radiographic imaging device 140 is a fluoroscopy system that captures radiographic imaging data in real time. In those embodiments, the method 2100 is a dynamic process where radiographic imaging data are not only obtained in the beginning but also throughout the performance of the method 2100. In fact, in some examples, the radiographic imaging data are displayed at steps 2155 and 2165 are displayed in real time. This allows the physician or clinician to monitor the position and orientation of the imaging assembly 102 in real time, facilitating effective and trouble-free catheterization and intra-cardiac examination procedures.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An ultrasound imaging system, comprising:
a flexible elongate member comprising a proximal portion and a distal portion, the distal portion configured to be positioned within a body of a patient,
an ultrasound imaging element disposed at the distal portion of the flexible elongate member and configured to obtain ultrasound data from within the body of the patient;
a handle coupled to the proximal portion of the flexible elongate member, wherein the handle comprises an accelerometer configured to generate orientation data corresponding to the ultrasound imaging element; and
a computing device in communication with the handle and the ultrasound imaging element, wherein the computing device is configured to receive the ultrasound and orientation data and co-register the received data to radiographic imaging data.

2. The ultrasound imaging system of claim 1, wherein the accelerometer is a dual-axis accelerometer.

3. The ultrasound imaging system of claim 1, wherein the ultrasound imaging element comprises a transducer array.

4. The ultrasound imaging system of claim 1, wherein the handle comprises a first actuator, upon activation, configured to deflect the distal portion of the flexible elongate member along a first plane.

5. The ultrasound imaging system of claim 4, wherein the handle further comprises a second actuator, upon actuation, configured to deflect the distal portion of the flexible elongate member along a second plane not parallel to the first plane.

6. A medical imaging system, comprising:
an ultrasound imaging device configured to obtain ultrasound imaging data of an area of interest within a body of a patient, the ultrasound imaging device including a flexible elongate member and a handle, wherein a distal portion of the flexible elongate member is sized and shaped to be inserted into the body and placed in the area of interest, wherein the handle includes an accelerometer configured to determine an orientation of the flexible elongate member; and
a computing device in communication with the ultrasound imaging device and a radiographic imaging device, the computing device configured to:
receive radiographic imaging data of the area of interest from the radiographic imaging device;
determine a position of the ultrasound imaging device using the radiographic imaging data;
receive the orientation of the flexible elongate member from the accelerometer;
receive ultrasound imaging data from the ultrasound imaging device;
co-register the ultrasound imaging data and the radiographic imaging data; and
output the co-registered ultrasound imaging data and radiographic imaging data to a display.

7. The medical imaging system of claim 6, wherein the computing device is further configured to control the ultrasound imaging device and the radiographic imaging device.

8. The medical imaging system of claim 6, wherein the computing device is further configured to overlay the ultrasound imaging data over the radiographic imaging data co-registered with the ultrasound imaging data and output to the display the ultrasound imaging data overlaid over the radiographic imaging data co-registered with the ultrasound imaging device.

9. The medical imaging system of claim 6, further comprising the radiographic imaging device.

10. The medical imaging system of claim 6, further comprising the display.

11. The medical imaging system of claim 6, wherein the ultrasound imaging device is intra-cardiac echocardiography (ICE) device.

12. The medical imaging system of claim 6, wherein the ultrasound imaging device is a trans-esophageal echocardiography (TEE) device.

13. The medical imaging system of claim 6, wherein the ultrasound imaging device is an intravascular ultrasound (IVUS) device.

14. The medical imaging system of claim 6, wherein the accelerometer is a dual-axis accelerometer.

15. The medical imaging system of claim 6, wherein the accelerometer is a triple-axis accelerometer.

16. A method for displaying ultrasound imaging data and radiographic imaging data with respect to an area of interest within a body of a patient, comprising:
obtaining the radiographic imaging data of the area of interest from outside of the body by a radiographic imaging device while an ultrasound imaging device is placed within the area of interest, wherein the ultrasound imaging device comprises a handle, the handle including an accelerometer configured to determine a orientation of the ultrasound imaging device;
determining a position of the ultrasound imaging device based on the radiographic imaging data;
receiving the orientation of the ultrasound imaging device from the accelerometer;
obtaining, by the ultrasound imaging device, the ultrasound imaging data of the area of interest from within the body;
co-registering the ultrasound imaging data and the radiographic imaging data based on the position and rotational orientation of the ultrasound imaging device; and
displaying the co-registered ultrasound imaging data and radiographic imaging data on a display.

17. The method of claim 16, further comprising overlaying ultrasound imaging data over the radiographic imaging data co-registered with the ultrasound imaging data.

18. The method of claim 16, further comprising displaying the ultrasound imaging data overlaid over the radiographic imaging data co-registered with the ultrasound imaging data.
